(19) United States
(12) Patent Application Publication
(10) Pub. No.: US 2026/0162263 A1
(43) Pub. Date: Jun. 11, 2026

(54) DEEP ULTRAVIOLET IMAGING AND IDENTIFICATION OF SUSPICIOUS OR CANCEROUS CELLS

(71) Applicant: Arizona Board of Regents on Behalf of the University of Arizona, Tucson, AZ (US)

(72) Inventor: Rongguang Liang, Tucson, AZ (US)

(21) Appl. No.: 18/975,660

(22) Filed: Dec. 10, 2024

(51) Int. Cl. G06T 7/00; A61B 10/02; G02B 21/16; G02B 21/36; G06T 7/62; G06V 10/82; G06V 20/69

(52) U.S. Cl. CPC G06T 7/0014; A61B 10/02; G02B 21/16; G02B 21/365; G06T 7/62; G06V 20/693; G06V 20/698; A61B 2010/0216; G06T 2207/10056; G06T 2207/20084; G06T 2207/30024; G06V 10/82; G06V 2201/03

(57) ABSTRACT

Methods, devices and system for imaging and analysis of cells of a sample without staining are described. One example method for assessing a presence of cancerous or suspicious regions includes obtaining a sample from a patient's oral cavity or cervix, spreading the sample onto a microscope slide, transporting the slide to a microscope, utilizing a deep ultraviolet light source, with wavelengths smaller than 300 nm, to obtain one or more images of the sample by the microscope, receiving and processing, at a processor, signals associated with the one or more images to: determine one or more geometrical characteristics of one or more cells of the sample, compare the one or more geometrical characteristics to one or more references, and based on comparison of the one or more geometrical characteristics to the one or more references, identify the one or more cells as one of a benign, cancerous or suspicious cell.

### TECHNICAL FIELD

The technology in this patent document relates to methods and devices for identifying normal, cancerous and precancerous cells.

### BACKGROUND

Early detection of various cancers is crucial for improving both the survival rate and quality of life for cancer patients. It is, therefore, important to develop accessible devices and methodologies for screening and triage and to quickly determine the presence of malignant or suspicious regions.

### SUMMARY

The disclosed embodiments relate to systems, devices and methods that leverage deep ultraviolet imaging techniques to enable imaging and analysis of cellular and nuclear architecture of cells without staining that delineate and identify normal, cancerous and precancerous cells or regions of a sample.

One example method for assessing a presence of cancerous or suspicious regions includes using a brush to obtain a sample from a patient's oral cavity or cervix, spreading the sample onto a microscope slide, transporting the slide to a microscope, utilizing a deep ultraviolet (UV) light source, having illumination wavelengths smaller than 300 nm, to obtain one or more images of the sample by the microscope, receiving and processing, at a processor, signals associated with the one or more images to: determine one or more geometrical characteristics of one or more cells of the sample, compare the one or more geometrical characteristics to one or more references, and based on comparison of the one or more geometrical characteristics to the one or more references, identify the one or more cells as one of a benign, cancerous or suspicious cell.

### BRIEF DESCRIPTION OF THE DRAWINGS

FIG. 1 illustrates the steps associated with two typical cervical screening operations of existing systems.

FIG. 2 illustrates a comparison of steps associated prior cancer screening operations and a procedure in accordance with an example embodiment.

FIG. 3 illustrates example images of cervical cells obtained based on deep UV imaging in accordance with example embodiments and their comparison to images obtained via liquid-based cytology.

FIG. 4A illustrates an example system in accordance with an example embodiment that uses deep UV imaging.

FIG. 4B illustrates an example image of cells obtained using the configuration of FIG. 4A.

FIG. 4C illustrates an example image of a healthy sample obtained from inside the oral cavity of a patient using the configuration of FIG. 4A.

FIG. 4D illustrates an example of an unstained deep ultraviolet image and an example of a stained visible image of a suspicious sample obtained using the configuration of FIG. 4A.

FIG. 5 illustrates a set of operations that can be carried out to assess a presence of cancerous or suspicious regions in accordance with an example embodiment.

FIG. 6 illustrates a set of operations that can be carried out to assess a presence of cancerous or suspicious regions in accordance with another example embodiment.

### DETAILED DESCRIPTION

In the sections that follow, oral and cervical cancers are used as examples to illustrate the various features and benefits of the disclosed embodiments.

Oral and oropharyngeal squamous cell carcinomas (OSCCs) together rank as the sixth most common cancer worldwide, over 640,000 new cases of oral cancer occur worldwide each year. In the United States, more than 50 thousand oral cancer cases and 11 thousand deaths occur annually. Low resource populations have the highest rates of oral cancer in the U.S., and a higher prevalence of oral potentially malignant lesions (OPML).

Driven by disparities in access to timely diagnosis, and care, individuals from low resource settings (LRS) have considerably poorer outcomes for oral cancer than others. The poor survival rate in LRS is mainly due to late detection and the resultant progression of disease to an advanced stage at diagnosis. The patients often present to the specialist with advanced stage disease, when the cancer has already spread from the oral cavity to the neck and distant sites. Early detection of oral cancer is effective in reducing morbidity and mortality.

The primary factor influencing survival outcomes is the stage of cancer at the time of the patient's first visit. The delays in diagnosing OSCC stem from a multifaceted set of factors that include patient-related issues, healthcare providers, infrastructure, and the broader health systems. Therefore, enhancing our capacity to identify potentially malignant and early-stage malignant changes could significantly boost overall survival rates.

Consequently, it is imperative to develop an accessible tool for screening and triage and to quickly determine the presence of malignant or suspicious regions.

Suspicious lesions are often categorized as OPML or cancer. In cases of OPML, the decision to biopsy is usually based on the presence of dysplasia. The gold standard for diagnosis has always been the tissue biopsy, known for its definitive, accurate, and reliable results. However, patients often express reluctance towards oral or oropharyngeal mucosal biopsies due to the invasive nature of the procedure, resulting in low compliance. This highlights the demand for a less invasive biopsy technique that maintains accuracy in detecting suspicious lesions and encourages patient compliance. An ideal solution would be a mobile tool capable of delineating and quantifying dysplasia without the need for standard invasive biopsy and conventional Hematoxylin and Eosin (H/E) staining.

Similarly, cervical cancer is diagnosed in about 13,000 women in the US annually, leading to 4,000 deaths. Early detection of cervical cancer is crucial as the 5-year relative survival rate drops to 59% if cancer has already spread to nearby tissues, organs, or regional lymph nodes, whereas it is 92% if it is diagnosed at an early stage. Hence, screening for cervical cancer is essential to prevent its development. Two types of screening tests are available for cervical cancer: Pap test and HPV test. While the Pap test checks for abnormal or cancerous cells in a sample, the HPV test looks for the presence of HPV infection. However, the Pap test faces challenges in LRS, including (1) it is a time-consuming process of slide preparation, staining, imaging, and interpretation; (2) it requires laboratory analysis and trained professionals to identify abnormalities or precancerous lesions.

Cervical cancer screening has made significant strides in reducing both new cases and deaths from the disease. However, there is a growing concern regarding the increasing percentage of women in the United States who are overdue for cervical cancer screening, and the underlying reasons for this trend remain unclear.

Brush cytology holds promise in bridging the diagnostic gap in the early detection of various cancers, such as oral and cervical cancers. This technique offers a minimally invasive approach to diagnosing dysplasia and early-stage carcinoma, particularly in asymptomatic patients or those with mild symptoms who may not require an immediate biopsy. However, the current application of brush cytology as a screening tool faces limitations and delays. The process involves collecting a cell sample and sending it to a laboratory for analysis, with results taking several days. This delay reduces its effectiveness for immediate cancer screening, especially in low resource settings, where there are limited laboratory facilities for cytological analysis.

The disclosed embodiments leverage deep ultraviolet imaging techniques to enable imaging and analysis of cellular and nuclear architecture of epithelial cells without staining and may help to delineate and quantify dysplasia faster than conventional H/E. The specific range of deep-UV (DUV) light (200-280 nm), is particularly effective due to its strong absorption by biological molecules, resulting in high specificity and contrast in label-free imaging. This absorption is mainly attributed to nucleic acids and proteins, facilitating the extraction of detailed information on cellular architecture and biochemistry. Consequently, DUV light's distinctive absorption characteristics enables the generation of highly detailed and quantitative images of biological samples.

This approach disclosed herein can be considered a stain-free or label-free brush biopsy technique, which facilitates cancer screening, detection, and diagnosis. In some embodiments, a DUV Fourier Ptychographic Microscope (DUV-FPM) is used to implement stainless cancer screening tests. The disclosed technology will enable a stainless screening to be performed directly at the point-of-care clinics, eliminating the need to send samples to a laboratory. In some embodiments, a deep-learning image classification algorithms is used that analyzes the gathered data. The patients are provided with screening results in just a few minutes, thus making it a valuable tool for cancer screening, such as oral and cervical cancer screening, particularly in low-resource settings.

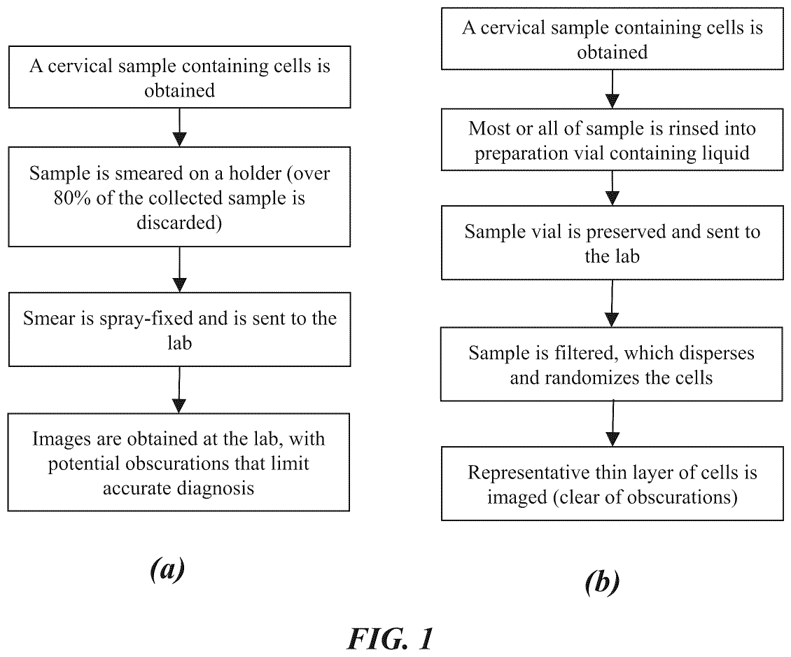

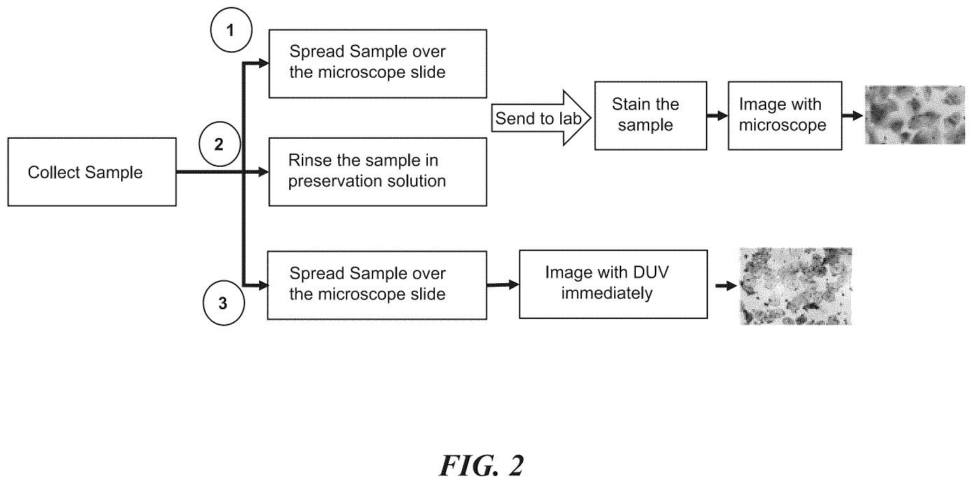

FIG. 1 illustrates the steps associated with two typical cervical screening operations of the existing systems: Conventional Pap Smear (panel (a)) and Liquid-Based Cytology (panel (b)). In the pap smear test, after obtaining a sample, a slide prepared wherein over 80% of the sample is discarded. This means that abnormal cells can be discarded and not imaged. The smeared sample is then spray-fixed and sent to a lab. However, the sample that is received by the lab includes cells that are dried out and clumped together, which may obscure the view of normal cells. In the liquid-based technique (panel (b)), the samples are rinsed and placed into a vial with a liquid therein, and sent to the lab. A filtration process disperses and randomizes the cells, producing a thin layer of cells that is clear of obscuring elements, which can improve the detection results compared to pap smear test. However, examination of randomly positioned cells may not provide the proper insight or information that is needed for a proper diagnosis. Furthermore, in both testing procedures, the samples must be sent to the laboratory, where the sample of stained and imaged with a microscope. This is further illustrated in FIG. 2 by the paths labeled as 1 and 2. In contrast, the path labeled as 3, illustrates a procedure in accordance with an example embodiment of the present application, where the samples obtained via, for example a brush in a pap test, are spread over a microscope slide and imaged immediately using deep UV light imaging. The deep UV image is analyzed for detection and diagnosis of cancer and/or suspicious cells.

By eliminating the logistical challenges associated with sample transportation and laboratory processing, the screening process is streamlined and improved to allow timely and accurate diagnostics. Notably, not only the need for staining the specimens is eliminated, but the time-consuming and costly step of shipping samples to the lab is also not needed. Immediate testing after specimen collection enables patients to receive results rapidly (e.g., in less than 10 minutes), rather than waiting for days.

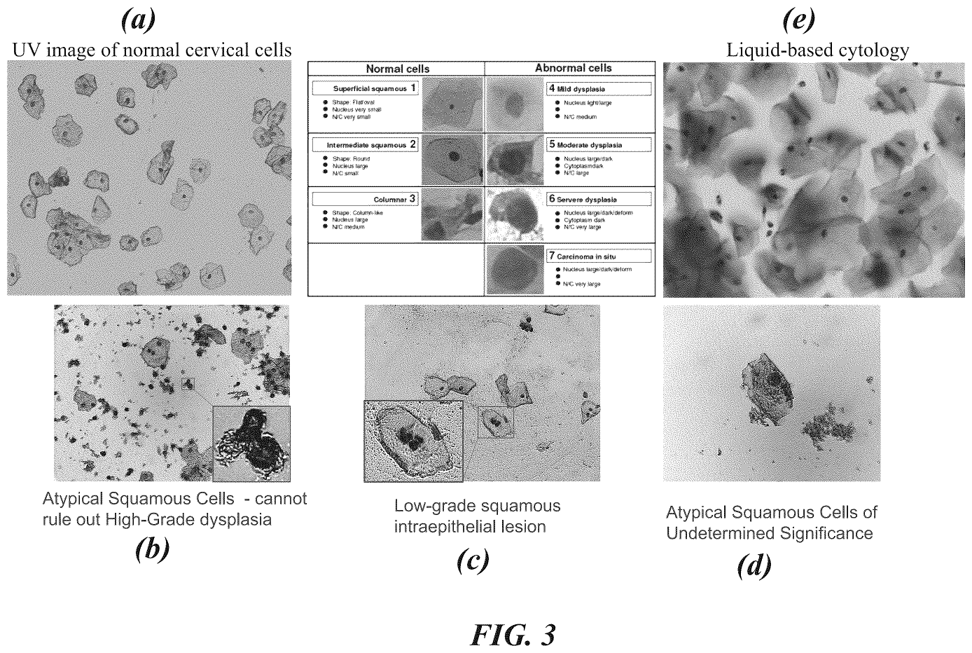

Deep UV light (<300 nm) is effective in detecting cancer cell because the deep UV transmittances in cancer cells is significantly lower than in non-cancer cells. As a result, UV imaging can reveal abnormalities in the nuclei of cancer cells that visible light cannot detect, and deep UV image of cervical and oral cavity cells can provide sufficient details for detection of abnormal cells. FIG. 3 illustrates example images of cervical cells obtained based on deep UV imaging and their comparison to images obtained via liquid-based cytology. Panel (a)-(d) illustrate example cell images obtained by using deep UV; panel (a) shows normal cervical cells; panel (b) shows atypical squamous cell imaged using deep UV, which may be high-grade dysplasia; panel (c) illustrates low-grade squamous intraepithelial lesions; and panel (d) illustrates atypical squamous cells of undetermined significance. The table in the middle of FIG. 3 shows various characteristics of normal and abnormal cells. In panel (e), an image obtained using liquid-based cytology is provided for comparison.

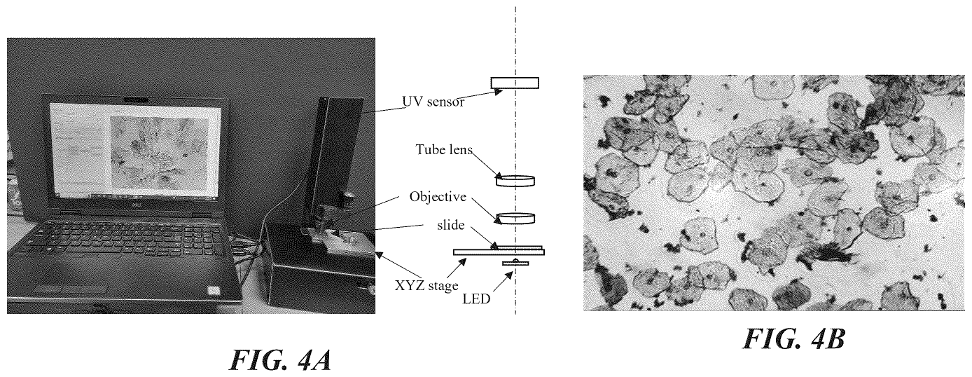

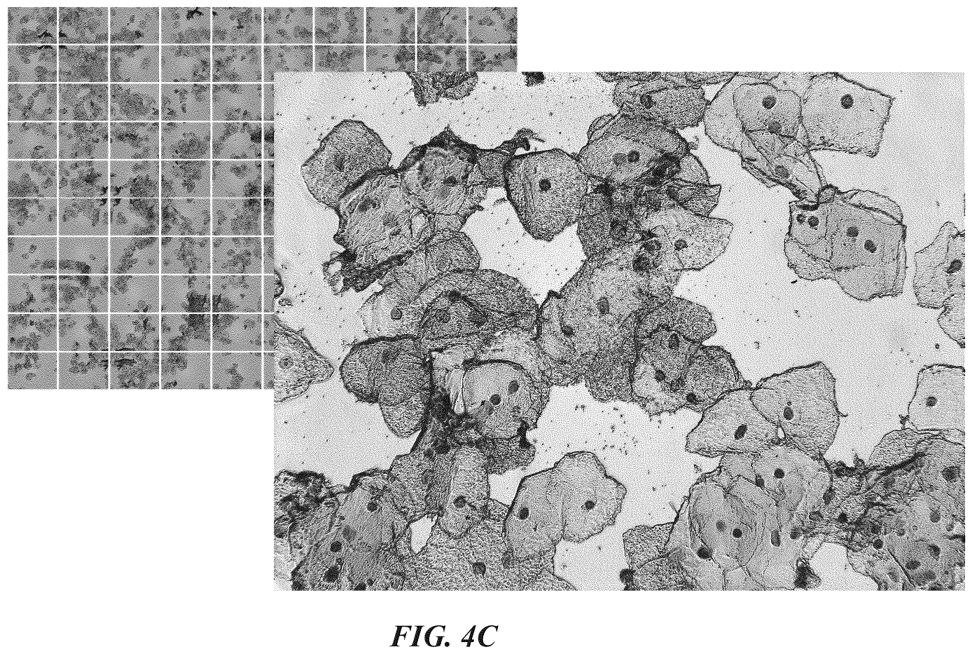

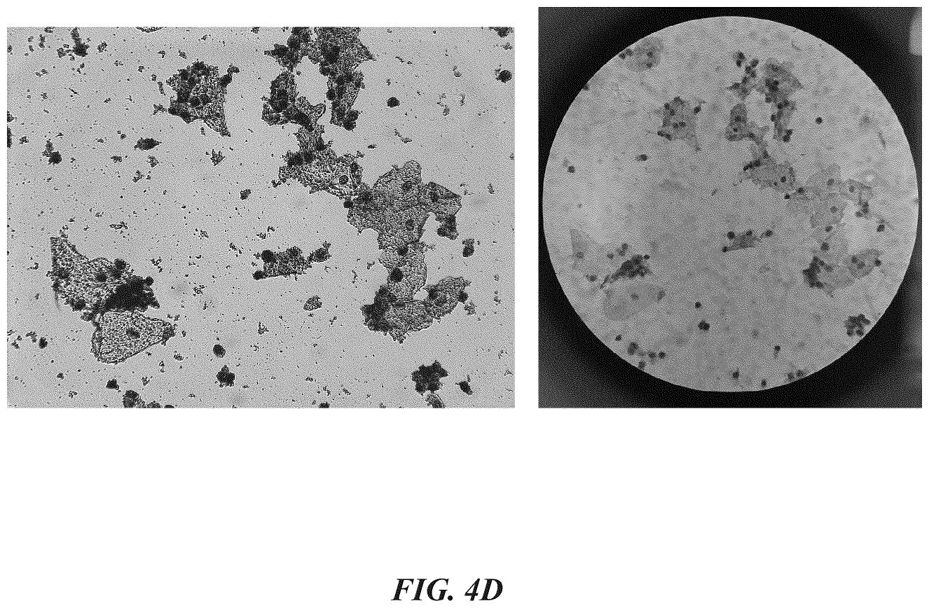

FIG. 4A illustrates an example system in accordance with an example embodiment that uses deep UV imaging, and an example image of normal cells is shown in FIG. 4B. In the system of FIG. 4A, deep UV light source (e.g., an LED) illuminates the sample (e.g., positioned on a slide) from the bottom. In one example system, a UV objective with a numerical aperture (NA) of 0.4 and a UV enhanced CMOS sensor is used. An XYZ stage for holding and translating the sample can be used, and tube lens may be positioned between the objective and the sensor. The system in panel (a) may be implemented at a cost of less than $1,000 with a low-cost CMOS sensor, compared to a commercial laboratory system, which may cost in excess of $30,000. In FIG. 4B, an image of a cervical sample is shown that includes an abundance of crucial details necessary for accurate diagnosis, including the shape, size, and appearance of the cervical cells. These characteristics play a significant role in assessing the health and condition of the cells, aiding in the identification of any potential abnormalities. FIG. 4C shows an image of a healthy sample obtained from inside the oral cavity of a patient using the system of FIG. 4A, which similarly includes sufficient details for identification and classification of sample boundaries and nuclei. In contrast, FIG. 4D shows unstained deep UV image (left) and stained visible image (right) of a suspicious sample obtained, where cells with abnormal boundaries and clumped nuclei are observed.

In some embodiments, the method for cancer screening starts by collecting a sample from a patient using a collection tool, such as a brush. For example, a sample from the cervix or from the oral cavity is obtained, and transferred (e.g., smeared) to a microscope slide. The sample can then be immediately images using a simple microscope set up that is connected to a computing device (e.g., a laptop, a tablet, a mobile phone, etc.) that is capable of receiving image data from a sensor (e.g., a CMOS sensor or detector) associated with the microscope. The signals received at the computing device can be communicated via a wired or wireless interface using an appropriate protocol. The computing device, in some embodiments, may be a connected devices that can communicate with an external device, database, or network such as the Internet or the cloud. The computing device can perform various processing operations on the received data to display images associated with the collected sample on a screen and/or to identify various types of cells (including benign, suspicious and cancerous cells).

In some embodiments, the processing operations include identifying one or more cells associated with the collected sample as a benign, a suspicious or a cancerous cell based on the size/shape of the cell nucleus and the size/shape of the cell boundary. For instance, cancerous or suspicious cells can be larger and/or have irregular boundaries compared to benign cells. In one example, the identification can be carried out based on a nucleus-to-cell size ratio obtained from processing the image of the cell, such as the ratio of diameters or areas of the nucleus and the cell. In some embodiments, identification is carried out by comparing the ratio to a ratio associated with a benign cell obtained, for example, from a database that contains such information for the general population. In some embodiments, similar comparisons can be made to known ratios associated with cancerous or suspicious cells (e.g., obtained from a database) to assess whether the cell is cancerous, suspicious, or benign. For example, if the nucleus-to-cell size ratio is less than a first reference threshold, the cell is identified as benign; if the ratio is greater than a second reference ratio, the cell is identified as cancerous; and if the ratio has a value between the first and second reference ratios, the cell is identified as suspicious.

In some embodiments, geometric characteristics, such as area, diameter, shape and the like, of the nucleus and/or the cell can be compared directly to those associated with a normal cell. In some embodiments, the identification of benign versus cancerous versus suspicious cells can be carried out based on the irregularity (e.g., deviation from a circular or elliptical shape) of the nucleus and/or the cell. In some embodiments, identification of benign versus cancerous versus suspicious cells can be carried out based on the density of one or more regions of the cells based on the intensity of the images. Notably, the density and intensity of specific cellular regions, particularly the nucleus and cytoplasm, can be advantageously used to improve distinguishing the benign, suspicious, and cancerous cells because they reflect key structural and compositional changes associated with malignancy. These changes arise from the cellular and genetic alterations that occur as cells progress from normal to precancerous and cancerous states. These differences in density and intensity reflect fundamental alterations in cellular architecture and metabolism, making them reliable diagnostic markers for identifying benign, suspicious, and malignant cells in pap smear image analysis. Automated techniques leverage these features to ensure accurate and consistent classification, improving early detection of cervical cancer.

It should be noted that in some embodiments only black and white images—as opposed to color images—can be used to identify the cells, thus improving both the storage and processing speed of identification since color information need not be processed.

In some embodiments, processing of the information associated with the sample can be done at least in-part using an artificial intelligence system comprising neural networks. For example, a deep learning module can detect each cell and examine the detected cells from the images of label-free cell samples obtained with the deep UV imaging, for characteristics including shape, size, nucleocytoplasmic ratio, and intensity variations in both cells and nuclei. Then classification algorithm, such as Multilayer Perceptron (MLP) network, can be developed for case-wise classification to distinguish between non-dysplasia, dysplasia, and cancer.

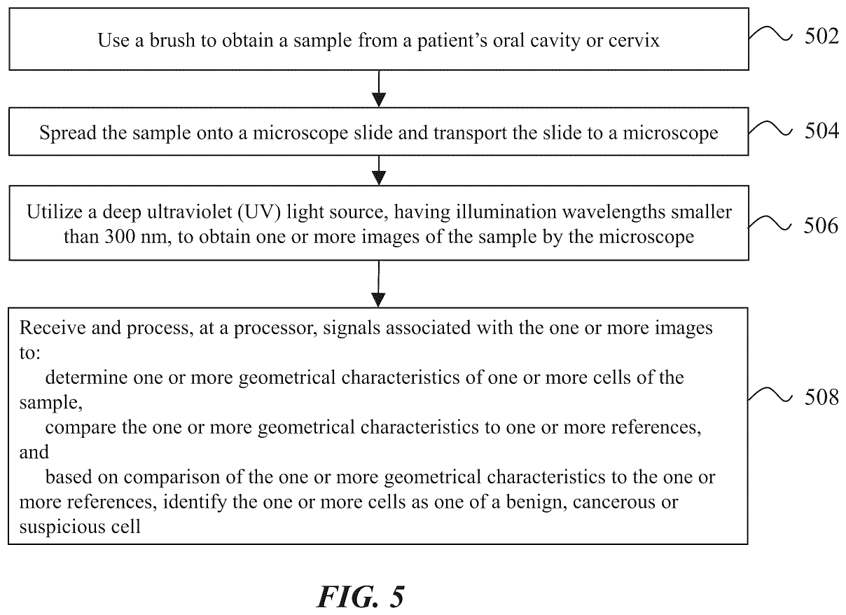

FIG. 5 illustrates a set of operations that can be carried out to assess a presence of cancerous or suspicious regions in accordance with an example embodiment. At 502, using a brush, a sample from a patient's oral cavity or cervix is obtained. At 504, the sample is spread onto a microscope slide which is then transported to a microscope. For example, the sample or a portion thereof may be transferred to a slide. At 506, utilizing a deep ultraviolet (UV) light source, having illumination wavelengths smaller than 300 nm, one or more images of the sample are obtained by the microscope. At 508, at a processor, signals associated with the one or more images are received and processed to (1) determine one or more geometrical characteristics of one or more cells of the sample, (2) compare the one or more geometrical characteristics to one or more references, and (3) based on comparison of the one or more geometrical characteristics to the one or more references, identify the one or more cells as one of a benign, cancerous or suspicious cell.

In one example embodiment, the one or more geometrical characteristics include a cell diameter and nucleus diameter, and the one or more cells are identified as the benign, cancerous or suspicious cell based on comparison of a ratio of the nucleus diameter to the cell diameter to one or more reference ratios indicative of a benign, cancerous or suspicious cell. In another example embodiment, the one or more geometrical characteristics include a cell size and nucleus size, and the one or more cells are identified as the benign, cancerous or suspicious cell based on comparison of a ratio of the nucleus size to the cell size with one or more reference ratios indicative of a benign, cancerous or suspicious cell. In still another example embodiment, the one or more geometrical characteristics include a nucleus shape, and the one or more cells are identified as the benign, cancerous or suspicious cell based on comparison of a cell shape to one or more a reference shapes indicative of a benign, cancerous or suspicious cell. In yet another example embodiment, the one or more geometrical characteristics include one or more irregularity measures associated with the one or more cells or one or more nuclei of the one or more cells, and the one or more cells are identified as the benign, cancerous or suspicious cell based on comparison of the one or more irregularity measures to one or more reference irregularity measures indicative of a benign, cancerous or suspicious cell.

According to another example embodiment, processing the signals associated with the one or more images further includes determining one or more intensity values associated with one or more regions of the one or more cells, and using the one or more intensity values for identification of the one or more cells as one of the benign, cancerous or suspicious cell. In one example embodiment, the operations include using the one or more intensity values to determine an opacity level, a transparency level, or a density of the one or more regions. In another example embodiment, the method includes using the density of the one or more regions to determine a structural or compositional change associated with the one or more cells indicative of a progression of a malignancy associated with the one or more cells.

In still another example embodiment, identification of the one or more cells as one of a benign, cancerous or suspicious cell is determined based on a combination of the following characteristics obtained from the one or more images: a shape of the one or more cells, a size of a nucleus of the one or more cells, a relative size of the one or more cells with respect to a nucleus of the one or more cells, an irregularity measure associated with the one or more cells or a nucleus of the one or more cells, or a density of a nucleus of the one or more cells. In yet another example embodiment, the one or more images are black-and-white images.

In one example embodiment, processing the signals associated with the one or more images includes using a neural network engine to classify one or more regions of the cell. In another example embodiment, the neural network engine comprises a deep learning module configured to process the one or more images, or portions thereof, to conduct a classification to distinguish between non-dysplasia, dysplasia, and cancer classifications based at least in part on one or more of a shape, a size, a nucleocytoplasmic ratio, or an intensity variation associated with the one or more cells or corresponding nuclei. In still another example embodiment, the neural network engine uses a multilayer perceptron network to conduct the classification. In yet another example embodiment, the one or more images are obtained from the sample that is not stained or treated with a liquid.

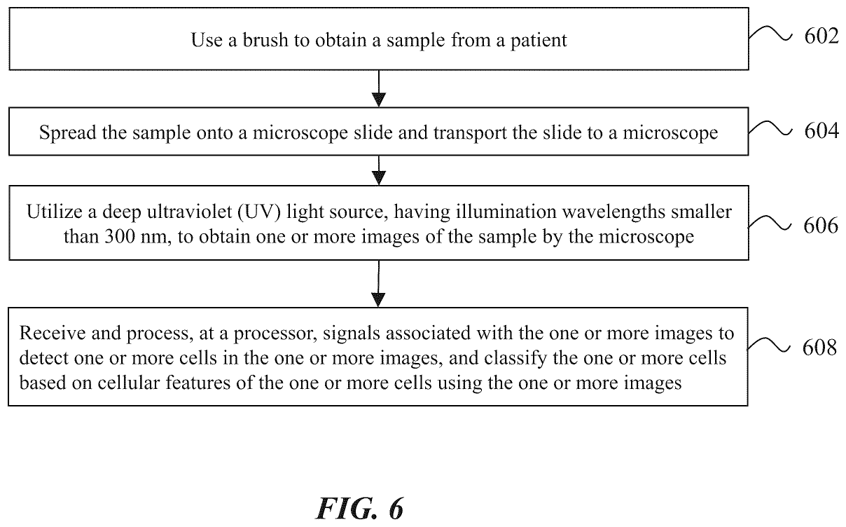

FIG. 6 illustrates a set of operations that can be carried out to assess a presence of cancerous or suspicious regions in accordance with another example embodiment. At 602, a sample from a patient is obtained. At 604, the sample is spread onto a microscope which is then transported to a microscope. The microscope can be collocated with the patient in the same office or building, for example. At 606, utilizing a deep ultraviolet (UV) light source, having illumination wavelengths smaller than 300 nm, one or images of the sample are obtained by the microscope. At 608, at a processor, signals associated with the one or more images are received and processed to: detect one or more cells in the one or more images, and classify the one or more cells based on cellular features of the one or more cells using the one or more images.

Another aspect of the disclosed embodiments relates to a system for assessing a presence of cancerous or suspicious regions that includes a microscope including a deep ultraviolet (UV) light source having illumination wavelengths smaller than 300 nm and positioned to illuminate a sample, an objective lens, and a UV image sensor configured to receive light from the sample in response to illumination of the sample by the deep UV light source. The system further includes a processor and a memory including instructions stored thereon; the processor is coupled to the microscope to receive signals associated with one or more images of the sample obtained by the UV image sensor. The instructions upon execution by the processor configure the processor to process the signals associated with the one or more images to: determine one or more geometrical characteristics associated with one or more cells, compare the one or more geometrical characteristics to one or more references, and based on comparison of the one or more geometrical characteristics to the one or more references, identify the one or more cells as one of a benign, cancerous or suspicious cell.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

It is understood that the various disclosed embodiments may be implemented individually, or collectively, in devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to receive electrical signals or information from the disclosed sensors (e.g., CMOS sensors), and to process the received information to produce images or other information of interest.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. The instructions may be stored on memory of a local processing device, or may be stored in a remote location, such as a remote server, a cloud sever, or other networked devices and environments. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes

Only a few implementations and examples are described and other implementations, enhancements and variations can be made based on what is described and illustrated in this patent document.

What is claimed is:

1. A method for assessing a presence of cancerous or suspicious regions, comprising:
using a brush to obtain a sample from a patient's oral cavity or cervix;
spreading the sample to a microscope slide;
transporting the slide to a microscope;
utilizing a deep ultraviolet (UV) light source, having illumination wavelengths smaller than 300 nm, to obtain one or more images of the sample by the microscope;
receiving and processing, at a processor, signals associated with the one or more images to:
determine one or more geometrical characteristics of one or more cells of the sample,
compare the one or more geometrical characteristics to one or more references, and
based on comparison of the one or more geometrical characteristics to the one or more references, identify the one or more cells as one of a benign, cancerous or suspicious cell.

2. The method of claim 1, wherein the one or more geometrical characteristics include a cell diameter and nucleus diameter, and the one or more cells are identified as the benign, cancerous or suspicious cell based on comparison of a ratio of the nucleus diameter to the cell diameter to one or more reference ratios indicative of a benign, cancerous or suspicious cell.

3. The method of claim 1, wherein the one or more geometrical characteristics include a cell size and nucleus size, and the one or more cells are identified as the benign, cancerous or suspicious cell based on comparison of a ratio of the nucleus size to the cell size to one or more reference ratios indicative of a benign, cancerous or suspicious cell.

4. The method of claim 1, wherein the one or more geometrical characteristics include a nucleus shape, and the one or more cells are identified as the benign, cancerous or suspicious cell based on comparison of a cell shape to one or more a reference shapes indicative of a benign, cancerous or suspicious cell.

5. The method of claim 1, wherein the one or more geometrical characteristics include one or more irregularity measures associated with the one or more cells or one or more nuclei of the one or more cells, and the one or more cells are identified as the benign, cancerous or suspicious cell based on comparison of the one or more irregularity measures to one or more reference irregularity measures indicative of a benign, cancerous or suspicious cell.

6. The method of claim 1, wherein processing the signals associated with the one or more images further includes determining one or more intensity values associated with one or more regions of the one or more cells, and using the one or more intensity values for identification of the one or more cells as one of the benign, cancerous or suspicious cell.

7. The method of claim 6, comprising using the one or more intensity values to determine an opacity level, a transparency level, or a density of the one or more regions.

8. The method of claim 7, comprising using the density of the one or more regions to determine a structural or compositional change associated with the one or more cells indicative of a progression of a malignancy associated with the one or more cells.

9. The method of claim 1, wherein identification of the one or more cells as one of a benign, cancerous or suspicious cell is determined based on a combination of the following characteristics obtained from the one or more images:
a shape of the one or more cells,
a size of a nucleus of the one or more cells,
a relative size of the one or more cells with respect to a nucleus of the one or more cells,
an irregularity measure associated with the one or more cells or a nucleus of the one or more cells, or
a density of a nucleus of the one or more cells.

10. The method of claim 1, wherein the one or more images are black-and-white images.

11. The method of claim 1, wherein processing the signals associated with the one or more images includes using a neural network engine to classify one or more regions of the cell.

12. The method of claim 11, wherein the neural network engine comprises a deep learning module configured to process the one or more images, or portions thereof, to conduct a classification to distinguish between non-dysplasia, dysplasia, and cancer classifications based at least in part on one or more of a shape, a size, a nucleocytoplasmic ratio, or an intensity variation associated with the one or more cells or corresponding nuclei.

13. The method of claim 12, wherein the neural network engine uses a multilayer perceptron network to conduct the classification.

14. The method of claim 1, wherein the one or more images are obtained from the sample that is not stained or treated with a liquid.

15. A method for assessing a presence of cancerous or suspicious regions, comprising:
obtaining a sample from a patient;
spreading the sample to a microscope slide;
transporting the slide to a microscope;
utilizing a deep ultraviolet (UV) light source, having illumination wavelengths smaller than 300 nm, to obtain one or images of the sample by the microscope; and
receiving and processing, at a processor, signals associated with the one or more images to:
detect one or more cells in the one or more images, and
classify the one or more cells based on cellular features of the one or more cells using the one or more images.

16. A system for assessing a presence of cancerous or suspicious regions, comprising:
a microscope comprising:
a deep ultraviolet (UV) light source having illumination wavelengths smaller than 300 nm and positioned to illuminate a sample,
an objective lens, and
a UV image sensor configured to receive light from the sample in response to illumination of the sample by the deep UV light source; and
a processor and a memory including instructions stored thereon, the processor coupled to the microscope to receive signals associated with one or more images of the sample obtained by the UV image sensor, wherein the instructions upon execution by the processor configure the processor to process the signals associated with the one or more images to:
determine one or more geometrical characteristics associated with one or more cells of the sample,
compare the one or more geometrical characteristics to one or more references, and
based on comparison of the one or more geometrical characteristics to the one or more references, identify the one or more cells as one of a benign, cancerous or suspicious cell.

17. The system of claim 16, wherein the one or more geometrical characteristics include a cell diameter and nucleus diameter, and the one or more cells are identified as the benign, cancerous or suspicious cell based on comparison of a ratio of the nucleus diameter to the cell diameter to one or more reference ratios indicative of a benign, cancerous or suspicious cell.

18. The system of claim 16, wherein the one or more geometrical characteristics include a cell size and nucleus size, and the one or more cells are identified as the benign, cancerous or suspicious cell based on comparison of a ratio of the nucleus size to the cell size to one or more reference ratios indicative of a benign, cancerous or suspicious cell.

19. The system of claim 16, wherein the one or more geometrical characteristics include one or more irregularity measures associated with the one or more cells or one or more nuclei of the one or more cells, and the one or more cells are identified as the benign, cancerous or suspicious cell based on comparison of the one or more irregularity measures to one or more reference irregularity measures indicative of a benign, cancerous or suspicious cell.

20. The system of claim 16, comprising a neural network engine having a deep learning module configured to process the one or more images, or portions thereof, to conduct a classification to distinguish between non-dysplasia, dysplasia, and cancer classifications based at least in part on one or more of a shape, a size, a nucleocytoplasmic ratio, or an intensity variation associated with the one or more cells or corresponding nuclei.
